(19) United States
(12) Patent Application Publication
(10) Pub. No.: US 2025/0197538 A1
(43) Pub. Date: Jun. 19, 2025

(54) NON-SILOXANE SENSORY MODIFIER

(71) Applicants: Dow Silicones Corporation, Midland, MI (US); Dow Global Technologies LLC, Midland, MI (US)

(72) Inventors: Fang Zhang, Midland, MI (US); Jordan C. Reddel, Midland, MI (US); Matthew E. Belowich, Midland, MI (US)

(21) Appl. No.: 18/849,833

(22) PCT Filed: Feb. 16, 2023
(86) PCT No.: PCT/US2023/013207

§ 371 Date: Sep. 23, 2024

Related U.S. Application Data
(60) Provisional application No. 63/328,471, filed on Apr. 7, 2022.

(51) Int. Cl. C08F 10/14; C08J 3/24

(52) U.S. Cl. CPC C08F 10/14; C08J 3/24

(57) ABSTRACT

A composition contains a crosslinked elastomer comprising polyolefin backbones crosslinked through polyether chains wherein the crosslinked elastomer has a carbon-silicon-carbon linkage between the polyolefin backbone and polyether chain and wherein the crosslinked elastomer is free of Si—O—Si bonds.

### FIELD OF THE INVENTION

The present invention is a crosslinked elastomer that is free of siloxane bonds, a composition comprising the crosslinked elastomer and a process for producing the crosslinked elastomer.

### INTRODUCTION

U.S. Pat. No. 9,999,586 ('586) teaches that silicone elastomers are considered one of the best sensory modifiers used in skin care formulations. '586 identifies the unique structure of silicone elastomers as a reason silicone elastomers have a skin feel unlike any silicone fluid or cationic quaternary compounds previously used as sensory modifiers. The feel of silicone elastomers has been described as “velvety, powdery, smooth and cushion feel.” Silicone elastomers are used in solvent-swollen pastes as sensory modifiers where the elastomer is solvent-swollen and blended to break up the elastomer into pieces to form a paste. Desirably, the pieces of the solvent-swollen elastomer have an average particle size in a range of one to 1,000 micrometers, preferably one to 100 micrometers, and more preferably one to 50 micrometers and even more preferably one to 25 micrometers. Smaller particles are more desirable for a smoother feel.

There is, however, a desire to identify sensory modifiers that provide a similar feel as silicone elastomers but with materials other than silicone because there is a chance that silicones can undesirably contain low molecular weight volatile silicones and cyclics.

U.S. Pat. No. 9,999,586 describes silicone modified polyolefins as possible alternatives to silicone elastomers for use as sensory modifiers for use in skin care formulations. The silicone modified polyolefins are sought to have similar sensory feel to silicone elastomers but contain polyolefin and silicone polymer components to reduce the amount of silicone polymer in the material. The silicone modified polyolefin still relies on silicone polymer to achieve the unique structure associated with the desired skin feel of the sensory modifier.

It is further desirable for the elastomer and pastes of the elastomers to be compatible with a variety of organic solvents used in cosmetic formulations so that the elastomer can be formulated in a variety of cosmetic formulations. Typical organic solvents include octyl methoxycinnamate, ethylhexyl salicylate, sunflower oil, squalane and mineral oil. Compatible elastomers and pastes form clear or hazy mixtures with a solvent while not being cloudy, opaque or phase separating from the solvent.

It is desirable to identify an elastomer that can be a sensory modifier with a similar skin feel to silicone elastomers commonly used in the cosmetic industry for sensory modifiers, but that does not require silicone. It is further desirable for the elastomer to have solvent compatibility with a variety of organic solvents used in cosmetic formulations.

### BRIEF SUMMARY OF THE INVENTION

The present invention offers an elastomer that can be a sensory modifier with a similar skin feel to silicone elastomers but that does not require silicone. The present invention can also provide an elastomer that can form a paste that is compatible with a variety of organic solvents at least as well as similar pastes of siloxane elastomers common in the cosmetic field.

The present invention is a result of discovering that crosslinking a polyolefin with polyether crosslinkers and connecting the polyolefin and polyether with a carbon-silicon-carbon (C—Si—C) linkage produces an elastomer that can be solvent swollen and mixed into a paste having a skin feel similar to pastes made with silicone elastomers. The combination of the C—Si—C linkage and the polyether crosslinking is thought to cause the particular skin feel. The unique bond angles produced by C—Si—C linkages is thought to contribute to a desirable morphology that promotes the desired skin feel. The polyether provides the desirable compatibility to the elastomer to allow formulating the elastomer in a variety of formulations and to provide desirably mobility between polyolefin backbone chains. Together, the C—Si—C linkage and polyether crosslinks are thought to result in an elastomer having a skin feel in solvent-swollen paste that is similar to paste made from silicone elastomers.

In a first aspect, the present invention is a composition comprising a crosslinked elastomer comprising polyolefin backbones crosslinked through polyether chains wherein the crosslinked elastomer has a carbon-silicon-carbon linkage between the polyolefin backbone and polyether chain and wherein the crosslinked elastomer is free of Si—O—Si bonds, which are necessary in a silicone.

In a second aspect, the present invention is a process for producing the composition of the first aspect, the process comprising providing a crosslinked elastomer by conducting a hydrosilylation reaction with a reaction composition comprising: (a) a hydrosilane functional polyolefin that is free of Si—O—Si bonds; (b) a polyether crosslinker comprising at least two terminal alkenyl groups and that is free of Si—O—Si bonds; (c) a hydrosilylation catalyst; and (d) an organic solvent.

The composition of the present invention is useful in a sensory modifier for skin care products.

### DETAILED DESCRIPTION OF THE INVENTION

Test methods refer to the most recent test method as of the priority date of this document when a date is not indicated with the test method number. References to test methods contain both a reference to the testing society and the test method number. The following test method abbreviations and identifiers apply herein: ASTM refers to ASTM International methods; EN refers to European Norm; DIN refers to Deutsches Institut für Normung; ISO refers to International Organization for Standards; and UL refers to Underwriters Laboratory.

Products identified by their tradename refer to the compositions available under those tradenames on the priority date of this document.

“Multiple” means two or more. “And/or” means “and, or as an alternative”. All ranges include endpoints unless otherwise indicated.

The term “interpolymer” refers to polymer prepared by the polymerization of at least two different types of monomers. The term interpolymer includes the term copolymer (which refers to polymers prepared from two different types of monomers) and polymers prepared from more than two different types of monomers.

For polymers herein, use the following gel permeation chromatography method to determine number-average molecular weight (“Mn_{(GPC)}”):

### Gel Permeation Chromotography Method for Molecular Weight

The chromatographic system consisted of a PolymerChar GPC-IR (Valencia, Spain) high temperature GPC chromatograph, equipped with an internal IR5 infrared detector (IR5). The autosampler oven compartment was set at 1600 Celsius, and the column compartment was set at 150° Celsius. The columns were one Agilent PLgel MIXED 7.5×50 mm, 20 μm linear mixed-bed guard column and four Agilent PLgel MIXED-A 7.5×300 mm, 20 μm linear mixed-bed columns. The chromatographic solvent was 1,2,4-trichlorobenzene, which contained 200 ppm of butylated hydroxytoluene (BHT). The injection volume used was 200 microliters, and the flow rate was 1.0 milliliters/minute.

Calibration of the GPC column set was performed using Agilent EasiCal Polystyrene standards (EasiCal PS-1 and EasiCal PS-2). Each EasiCal system consisted of two different spatulas supporting a mixture of 5 polymer standards (approximately 5 mg) to obtain 20 molecular weights points ranging from approximately 580 to 6,570,000 g/mole. Individual spatulas were added to septa-capped vials, sealed and loaded into the PolymerChar autosampler. PolymerChar Instrument Control Software was utilized to add 8 mL of solvent to each vial and the standards were dissolved for 15 minutes at 1600 Celsius under high-speed shaking prior to injection to the chromatography system. A third order polynomial was used to fit the nominal polystyrene standard peak molecular weights to obtain molecular weight equivalent calibration points at each chromatographic slice.

The total plate count of the GPC column set was performed with decane (3% v/v in TCB introduced via micropump). The plate count for the chromatographic system should be greater than 18,000 for the 4 Agilent “Mixed A” 30 cm 20-micron linear mixed-bed columns.

Samples were prepared in a semi-automatic manner with the PolymerChar Instrument Control Software, wherein the samples were weight-targeted at 2 mg/ml, and the solvent was added to a septa-capped sealed vial, via the PolymerChar high temperature autosampler. The samples were dissolved for two hours at 1600 Celsius under high-speed shaking.

The calculations of Mn(GPC), Mw(GPC), and MZ(GPC) were based on GPC results using the internal IR5 detector (measurement channel) of the PolymerChar GPC-IR chromatograph according to Equations 1-3. Using PolymerChar GPCOne™ software, the baseline-subtracted IR chromatogram at each equally-spaced data collection point (i) was converted to the polystyrene equivalent molecular weight, obtained from the narrow standard calibration curve, for the equivalent chromatographic data point (i). Equations 1-3 are as follows:

$$Mn ( GPC ) = \frac{{\sum }^{i} {IR}_{i}}{{\sum }^{i} ( {IR}_{i} / {M}_{{polystyrene}_{i}} )} \qquad ( EQ 1 )$$

$$Mw ( GPC ) = \frac{{\sum }^{i} ( {IR}_{i} * {M}_{{polystyrene}_{i}} )}{{\sum }^{i} {IR}_{i}} \qquad ( EQ 2 )$$

$$Mz ( GPC ) = \frac{{\sum }^{i} ( {IR}_{i} * {M}_{{polystyrene}_{i}}^{2} )}{{\sum }^{i} ( {IR}_{i} * {M}_{{polystyrene}_{i}} )} \qquad ( EQ 3 )$$

In order to monitor the deviations over time, a flowrate marker (3% v/v decane in solvent) was introduced into each sample, via a micropump controlled with the PolymerChar GPC-IR system. This flowrate marker (FM) was used to linearly correct the pump flowrate (Flowrate(nominal)) for each sample, by RV alignment of the respective decane peak within the sample (RV(FM Sample)), to that of the decane peak within the narrow standards calibration (RV(FM Calibrated)). Any changes in the time of the decane marker peak were then assumed to be related to a linear-shift in flowrate (Flowrate(effective)) for the entire run. After calibrating the system based on a flow marker peak, the effective flowrate (with respect to the narrow standards calibration) is calculated as Equation 4. Processing of the flow marker peak was done via the PolymerChar GPCOne™ Software. Acceptable flowrate correction is such that the effective flowrate should be within +1-0.7% of the nominal flowrate.

$$Flowrate ( effective ) = Flowrate ( nominal ) * ( RV ( F M Calibrated ) / RV ( F M Sample ) ) \qquad ( EQ 4 )$$

The composition of the present invention comprises a crosslinked elastomer comprising polyolefin backbones crosslinked through polyether chains and further characterized by having carbon-silicon-carbon (C—Si—C) linkages connecting the polyolefin backbones and polyether chains and by being free of siloxane (Si—O—Si) linkages.

The polyolefin backbone is a homopolymer or interpolymer that comprises interpolymerized monomers that are polymerized through carbon-carbon double bonds (C=C). Desirably, the polyolefin backbone comprises polymerized alkenes selected from a group consisting of alkenes having 2 or more, preferably 3 or more, 4, or more, 5 or more, 6 or more, 7 or more, even 8 or more while at the same time typically 10 or fewer, 9 or fewer, 8 or fewer, 7 or fewer, 6 or fewer carbon atoms. Most desirably, the polyolefin backbone comprises an interpolymer comprising polymerized ethylene and octene.

The polyolefin backbone further comprises an interpolymerized monomer that produces a pendant carbon chain comprising a carbon-silicon bond (C—Si) linking a silicon atom to the polyolefin backbone, preferably the pendant carbon chain is terminated with a C—Si. Crosslinking occurs through the Si of the C—Si bond, specifically with a C—Si—C linkage that includes the C—Si bond. The polyolefin can, for example, comprises an interpolymerized alkenylsilane such a 7-octenyldimethylsilane and/or 5-hexenyldimethylsilane. Crosslinking can then occur through the silicone hydride bond (Si—H) bond of the silane to produce a C—Si—C linkage between the backbone and polyether crosslinker.

Desirably, the polyolefin backbone is an interpolymer that comprises or consists of the following polymerized monomer segments: (a) 20 to 35 mol-percent of octene; (b) 65 to 80 mole-percent ethylene; and (c) 1 to 10 mole-percent of one or any combination of more than one of 7-octenyldimethylsilane and 5-hexenyldimethylsilane.

The polyether chain that crosslinks polyolefin backbones typically comprises polyether units selected from —R—O— groups where R is an alkylene having 2 or more, 3 or more 4 or more, 5 or more, 6 or more, even 7 or more carbon atoms and at the same time typically has 8 or fewer, 7 or fewer, 6 or fewer, 5 or fewer, 4 or fewer, even 3 or fewer carbon atoms. For example the polyether chain can comprise any one or any combination of more than one of polyethylene oxide, polypropylene oxide, and polybutylene oxide.

The polyether chain that crosslinks the polyolefin backbones typically contains 3 or more, 5 or more, 10 or more, 15 or more, even 20 or more —R—O— groups while at the same time generally contains 100 or fewer, 75 or fewer, 60 or fewer 50 or fewer, 45 or fewer, 40 or fewer, 30 or fewer, 25 or fewer, or even 20 or fewer —R—O— groups.

The crosslinked elastomer desirably comprises or consists of the following interpolymerized polymer segments:
(a) 20 to 35 mole-percent (mol %) of one or a combination of more than one of segment with the average chemical structure —CH₂CHR′—;
(b) 65 to 80 mol % —CH₂CH₂—; and
(c) 1 to 10 mole-percent of —CH₂CHA-;
   wherein mol % is relative to moles of interpolymerized polymer segments in the crosslinked elastomer; each R′ is independently in each occurrence selected from a group consisting of alkyl groups having one or more, 2 or more, 3 or more, 4 or more, 5 or more, even 6 or more carbon atoms while at the same time typically has 8 or fewer, 7 or fewer, even 6 or fewer carbon atoms; and A has the following average chemical structure (I):

—(CH₂)ₙSi(CH₃)₂—B-D (I)
and where:
   n is independently in each occurrence a value in a range of 4 to 6 and can be 4, 5 or 6;
   B is —(CH₂)₃O(RO)_{p′}(CH₂)₃—;
      where:
         each R is independently in each occurrence as defined above, and is preferably one or a combination of both alkylene group selected from —CH₂CH₂— and —CH₂CH(CH₃)— groups; and
         subscript p′ has a value of 15 or more, or 20 or more while at the same time 25 or less; and
   D is the silicon of a —Si(CH₃)₂(CH₂)ₙ— group of another A group such that component B is a crosslinking group binding two backbone groups consisting of the other interpolymerized polymer segments.

The composition of the present invention can further comprise a solvent with the crosslinked elastomer. The crosslinked elastomer can be swollen with the elastomer to form a solvent swollen elastomer. A solvent swollen elastomer is an elastomer that has imbibed solvent. Generally, a solvent swollen elastomer has a greater volume than the elastomer alone without the presence of solvent.

Typical solvents include hydrocarbons, esters, and ethers. Examples of suitable solvents include any one or combination of more than one selected from isohexadecane, isododecane, undecane, tridecane, dicaprylyl carbonate, isodecyl neopentanoate, and di-n-octylether.

Desirably, the composition comprises solvent at a concentration of 90 wt % or more, or even 95 wt % or more while at the same time typically 97 wt % or less based on weight of the composition.

The composition can comprise particulates of solvent swollen elastomer. Desirably, the composition comprises solvent swollen elastomer having an average particle size of one micrometer or more, 5 micrometers or more, 10 micrometers or more, 20 micrometers or more, 30 micrometers or more, 40 micrometers or more, 50 micrometers or more, 75 micrometers or more, 100 micrometers or more, 125 micrometers or more, 150 micrometers or more 175 micrometers or more, 200 micrometers or more, 225 micrometers or more, 250 micrometers or more, 275 micrometers or more, 300 micrometers or more, 325 micrometers or more, 350 micrometers or more, 375 micrometers or more, 400 micrometers or more, 425 micrometers or more, even 450 micrometers or more while at the same time 500 micrometers or less, 475 micrometers or less, 450 micrometers or less, 425 micrometers or less, 400 micrometers or less, 375 micrometers or less, 350 micrometers or less, 325 micrometers or less, 300 micrometers or less, 275 micrometers or less, 250 micrometers or less, 225 micrometers or less, 200 micrometers or less, 175 micrometers or less, 150 micrometers or less, 125 micrometers or less, 100 micrometers or less, 75 micrometers or less, 50 micrometers or less, 25 micrometers or less, 20 micrometers or less, or even 10 micrometers or less.

Determine average particle size of the solvent swollen elastomer particulates using the following Elastomer Particulate Sizing method. The Elastomer Particulate Sizing method is a laser diffraction method using a Mastersize 3000 (from Malvern Panalytical). Prepare samples of elastomer paste by placing 1.00 grams (g) of a solvent swollen elastomer into a 20 g dental mixing cup and mixing for 30 seconds at 3500 revolutions per minute (RPM) using a speedmixer. Add 0.5 g of isododecane and mix for 30 seconds at 3500 RPM. Add another 0.5 g of isododecane and mix for 30 seconds at 3500 RPM. Add another 1.0 g of isododecane and mix for 30 seconds at 3500 RPM. Add another 2.0 g of isododecane and mix for 30 seconds at 3500 RPM. Add another 2.0 g of isododecane and mix for 30 seconds at 3500 RPM. Add another 4.0 g of isododecane and mix for 10 seconds at 3500 RPM to obtain an elastomer paste—which is a solvent swollen elastomer comprising particulates of solvent swollen elastomer. Add a sample of the elastomer past into a hydro SV fluid cell for average particle size determination using the Masterizer 3000. Collect and analyze data using Mastersizer v. 3.81 software and assume non-spherical particles, material refractive index of 1.5 (standard olefin), a dispersant refractive index of 1.42 (isododecane) and test at an obscuration between 2 and 25%.

The composition can comprise sunscreen agents, colorants and/or sun care additives such as titanium dioxide, zinc oxide or a combination thereof at a concentration of up to 20 wt % of the composition weight.

The present invention also includes a process for preparing the composition of the present invention by providing a crosslinked elastomer by conducting a hydrosilylation reaction with a reaction composition comprising (a) a hydrosilane functional polyolefin that is free of Si—O—Si bonds; (b) a polyether crosslinker comprising at least two terminal alkenyl groups and that is free of Si—O—Si bonds; (c) a hydrosilylation catalyst; and (d) and organic solvent.

The hydrosilane functional polyolefin that is free of Si—O—Si bonds can be prepared according to the teaching of U.S. Pat. No. 6,624,254. The hydrosilane functional polyolefin forms the polyolefin backbone of the crosslinked elastomer and so can be characterized as described for the polyolefin backbone herein, above. Desirably, the hydrosilane functional polyolefin has a number average molecular weight (Mn(GPC)) of 15,000 or more, 20,000 or more, 25,000 or more, 30,000 or more, 35,000 or more, 40,000 or more, 45,000 or more, even 50,000 or more while at the same time desirably has a Mn(GPC) of 150,000 or less, 146,000 or less, 140,000 or less, 120,000 or less, 100,000 or less, 80,000 or less, 70,000 or less, 60,000 or less, 55,000 or less, 50,000 or less, 45,000 or less, even 40,000 or less.

The hydrosilane functional polyolefin is a random interpolymer (as opposed to a block interpolymer). Desirably, the hydrosilane functional polyolefin is a random interpolymer comprising, or consisting essentially of (apart from possible terminal groups on the interpolymer) the following interpolymerized segments: —(CH₂CHR′)—, —R″—, and —(CH₂CHG)-; where:
Each R′ can be the same or different relative to other R′ groups and each R′ is independently, in each occurrence, selected from alkyl groups having one or more, 2 or more, 3 or more, 4 or more, 5 or more, 6 or more, or even 7 or more carbon atoms while at the same time typically having 8 or fewer, 7 or fewer, 6 or fewer, 5 or fewer, 4 or fewer, 3 or fewer, or even 2 or fewer carbon atoms;
Each R″ can be the same or different relative to other R″ groups and each R″ is independently, in each occurrence, selected from alkylene having one or more, 2 or more, 3 or more, 4 or more, 5 or more, 6 or more, or even 7 or more carbon atoms while at the same time typically having 8 or fewer, 7 or fewer, 6 or fewer, 5 or fewer, 4 or fewer, 3 or fewer, or even 2 or fewer carbon atoms;
Each G can be the same or different relative to other G groups and each G is independently in each occurrence a group selected from those having the following average chemical structure: —R³SiR⁴₂H, where
   each R³ can be the same or different relative to other R³ groups and each R³ is independently selected from a group consisting of alkylene groups having one or more, 2 or more, 3 or more, 4 or more, 5 or more, 6 or more, even 7 or more while at the same time typically 8 or fewer, 7 or fewer, 6 or fewer, 5 or fewer, 4 or fewer, 3 or fewer, even 2 or fewer carbon atoms; and
   each R⁴ can be the same or different relative to other R⁴ groups and each R⁴ is independently in each occurrence selected from a group consisting of hydrogen and alkyl groups having one or more, 2 or more, 3 or more, 4 or more, 5 or more, 6 or more, even 7 or more while at the same time typically 8 or fewer, 7 or fewer, 6 or fewer, 5 or fewer, 4 or fewer, 3 or fewer, even 2 or fewer carbon atoms.

The —(CH₂CHR′)— segments are typically present in the hydrosilane functional polyolefin at a concentration of 20 mole-percent (mol %) or more, 25 mol % or more, or even 30 mol % or more while at the same time typically 35 mol % or less, 30 mol % or less, or even 25 mol % or less; with mol % relative to total moles of interpolymerized segments.

The —R″— segments are typically present in the hydrosilane functional polyolefin at a concentration of 65 mol % or more, 70 mol % or more, or even 75 mol % or more while at the same time typically 80 mol % or less, 75 mol % or less, or even 70 mol % or less; with mol % relative to total moles of interpolymerized segments.

The —(CH₂CHG)- segments are typically present in the hydrosilane functional polyolefin at a concentration of one mol % or more, 5 mol % or more, or even 7.5 mol % or more while at the same time typically 10 mol % or less, 7.5 mol % or less, or even 5 mol % or less; with mol % relative to total moles of interpolymerized segments.

The hydrosilane functional polyolefin can be an interpolymer of: (a) 20 to 35 mol % 1-octene; (b) 65 to 80 mol % of ethylene; and (c) one to 10 mol % of one or any combination of both of 7-octenyldimethylsilane and 5-hexenyldimethylsilane with mole-percent relative to total moles of 1-octene, ethylene, 7-octenyldimethylsilane and 5-hexeneyldimethylsilane; with mol % relative to total moles of (a), (b) and (c).

The polyether crosslinker contains comprise polyether units selected from —R—O— groups where R is an alkylene having 2 or more, 3 or more 4 or more, 5 or more, 6 or more, even 7 or more carbon atoms and at the same time typically has 8 or fewer, 7 or fewer, 6 or fewer, 5 or fewer, 4 or fewer, even 3 or fewer carbon atoms. Generally, the polyether crosslinker contains 3 or more, 5 or more, 10 or more, 15 or more, even 20 or more —R—O— groups while at the same time generally contains 100 or fewer, 75 or fewer, 60 or fewer 50 or fewer, 45 or fewer, 40 or fewer, 30 or fewer, 25 or fewer, or even 20 or fewer —R—O— groups.

The polyether crosslinker can be, for example, any one or any combination of more than one selected from a group consisting of bis-allyl propylene glycol, bis-allylethylene and propylene glycol, alpha, omega-unsaturated hydrocarbons such as those having a chemical formula CH₂=CH(CH₂)_{d}CH═CH₂ where subscript d has an average value of zero or more, and can be one or more, 2 or more, 3 or more, 4 or more, 5 or more, 6 or more, 7 or more, 8 or more, 9 or more, 10 or more, 11 or more, 12 or more, 13 or more, even 14 or more while at the same time is typically 20 or less, and can be 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, or even 4 or less.

The hydrosilylation catalyst can be any catalysts useful for catalyzing hydrosilylation reactions. Typically, the hydrosilylation catalyst is a platinum-based catalyst. Platinum-based hydrosilylation catalysts include compounds and complexes such as platinum (0)-1,3-divinyl-1,1,3,3-tetramethyldisiloxane (Karstedt's catalyst), H₂PtCl₆, di-μ-carbonyl di-π-cyclopentadienyldinickel, platinum-carbonyl complexes, platinum-divinyltetramethyldisiloxane complexes, platinum cyclovinylmethylsiloxane complexes, platinum acetylacetonate (acac), platinum black, platinum compounds such as chloroplatinic acid, chloroplatinic acid hexahydrate, a reaction product of chloroplatinic acid and a monohydric alcohol, platinum bis(ethylacetoacetate), platinum bis(acetylacetonate), platinum dichloride, and complexes of the platinum compounds with olefins or low molecular weight organopolysiloxanes or platinum compounds microencapsulated in a matrix or core-shell type structure. The hydrosilylation catalyst can be part of a solution that includes complexes of platinum with low molecular weight organopolysiloxanes that include 1,3-diethenyl-1,1,3,3-tetramethyldisiloxane complexes with platinum. These complexes may be microencapsulated in a resin matrix. The catalyst can be 1,3-diethenyl-1,1,3,3-tetramethyldisiloxane complex with platinum.

Typical solvents include hydrocarbons, esters, and ethers. Examples of suitable solvents include any one or combination of more than one selected from isohexadecane, isododecane, undecane, tridecane, dicaprylyl carbonate, isodecyl neopentanoate, and di-n-octylether

### EXAMPLES

### Hydrosilane Functional Polyolefin (HFP) Synthesis

The ethylene/octene/silane interpolymerizations were conducted in a 2 L autoclave batch reactor designed for ethylene homo-polymerizations and co-polymerizations. The reactor was equipped with electrical heating bands, and an internal cooling coil containing chilled glycol. Both the reactor and the heating/cooling system were controlled and monitored by a process computer. The bottom of the reactor was fitted with a dump valve, which emptied the reactor contents into a dump pot that was vented to the atmosphere.

All chemicals used for polymerization and the catalyst solutions were run through purification columns prior to use. The ISOPAR-E, 1-octene, ethylene, and the silane monomers were also passed through columns. Ultra-high purity grade nitrogen (Airgas) and hydrogen (Airgas) were used. The catalyst cocktail was prepared by mixing, in an inert glove box, the scavenger (MMAO-3A), activator (bis(hydrogenated tallow alkyl)methyl tetrakis(pentafluoro-phenyl)borate amine), and pre-catalyst (zirconium, dimethyl[[2,2′″-[1,3-propanediylbis(oxy-κO)]bis[3″,5,5″-tris(1,1-dimethylethyl)-5′-methyl[1,1′:3′,1″-terphenyl]-2′-olato-xO]](2-)]-(ACI)) with the appropriate amount of toluene, to achieve a desired molarity solution. The solution was then diluted with ISOPAR-E or toluene to achieve the desired quantity for the polymerization and drawn into a syringe for transfer to a catalyst shot tank.

In a typical polymerization, the reactor was loaded with ISOPAR-E, and 1-octene via independent flow meters. The silane monomer was then added via a shot tank piped in through an adjacent glove box. After the solvent/comonomer addition, hydrogen (if desired) was added, while the reactor was heated to a polymerization setpoint of 120° C. The ethylene was then added to the reactor via a flow meter, at the desired reaction temperature, to maintain a predetermined reaction pressure set point. The catalyst solution was transferred into the shot tank, via syringe, and then added to the reactor via a high pressure nitrogen stream, after the reactor pressure set point was achieved. A run timer was started upon catalyst injection, after which, an exotherm was observed, as well as a decrease in the reactor pressure, to indicate a successful run.

Ethylene was then added using a pressure controller to maintain the reaction pressure set point in the reactor. The polymerizations were run to a set ethylene uptake, after which, the agitator was stopped, and the bottom dump valve was opened to empty the reactor contents into dump pot. The pot contents were poured into trays, which were placed in a fume hood, and the solvent was allowed to evaporate overnight. The trays containing the remaining polymer were then transferred to a vacuum oven, and heated to 100° C., under reduced pressure, to remove any residual solvent. After cooling to ambient temperature, the polymers were weighed for yield/efficiencies, transferred to containers for storage, and submitted for analytical testing. Polymerization conditions are shown in Table 1. Polymer properties are shown in Table 2.

TABLE 1
Polymerization Conditions to produce HFP

| | Pre- | | | | Silane | | | |
|---|---|---|---|---|---|---|---|---|
| | catalyst | Ethylene | Ethylene | Octene | monomer | Solvent | H₂ | Run |
| | loading | loaded | uptake | Loaded | loaded | loaded | loaded | Time |
| Resin | (μmol) | (g) | (g) | (g) | (g) | (g) | (mmol) | (min) |
| HFP1 | 0.5 | 12.3 | 22.9 | 70.2 | 12.1 | 586.9 | 4.7 | 9.4 |
| HFP2 | 1.5 | 12.1 | 22.9 | 150.0 | 12.1 | 510.4 | 35.9 | 13.9 |

TABLE 2
Polymer Properties

| | Conventional GPC | | | Monomer Ratio Information | | | | | |
|---|---|---|---|---|---|---|---|---|---|
| Resin | Summary | | | Determined by ¹H NMR* | | | | DSC | |
| Description | Mn | Mw | | Ethylene | Octene | Silane | Silane | Tg | Tm |
| Resin | (g/mol) | (g/mol) | Mw/Mn | (mol %) | (mol %) | Type | mol% | (° C.) | (° C.) |
| HFP1 | 145990 | 374530 | 2.57 | 76.55 | 20.82 | ODMS^{A} | 2.62 | −63.3 | 38.1 |
| HFP2 | 37530 | 137400 | 2.21 | 68.47 | 29.68 | HDMS^{B} | 1.85 | −69.6 | −31 |

*Mol % based on total moles of monomers in polymer determined by ¹H NMR.
^{A}ODMS = 7-Octenyldimethylsilane.
^{B}HDMS = 5-Hexenyldimethylsilane.

Number average molecular weight (Mn), weight average molecular weight (Mw) and polydispersity (Mw/Mn) are determined by gel permeation chromatography (GPC) according to the Gel Permeation Chromotography Method for Molecular Weight presented herein above.

Monomer Ratio Information is determined by proton nuclear magnetic resonance spectroscopy (¹H NMR) according to the following method. Samples were dissolved, in 8 mm NMR tubes, in tetrachloroethane-d₂ (with or without 0.001 M Cr(acac)₃). The concentration was approximately 100 mg/1.8 mL. The tubes were then heated in a heating block set at 110° C. The sample tubes were repeatedly vortexed and heated to achieve a homogeneous flowing fluid. The ¹H NMR spectra were taken on a BRUKER AVANCE 600 MHz spectrometer, equipped with a 10 mm C/H DUAL cryoprobe. A standard single pulse ¹H NMR experiment was performed. The following acquisition parameters were used: 70 seconds relaxation delay, 90 degree pulse of 17.2 μs, 32 scans. The spectra were centered at 1.3 ppm, with a spectral width of 20 ppm. All measurements were taken, without sample spinning, at 110° C. The ¹H NMR spectra were referenced to “5.99 ppm” for the resonance peak of the solvent (residual protonated tetrachloroethane). For a sample with Cr, the data was taken with a “16 seconds relaxation delay” and 128 scans.

Glass transition temperature (Tg) and melt temperature (Tm) were determined using differential scanning calorimetry (DSC) according to the following method: Each sample (0.5 g) was compression molded into a film, at 5000 psi, 190° C., for two minutes. About 5 to 8 mg of film sample was weighed and placed in a DSC pan. The lid was crimped on the pan to ensure a closed atmosphere. Unless otherwise stated, the sample pan was placed in a DSC cell, and then heated, at a rate of 10° C./min, to a temperature of 180° C. for PE (230° C. for PP). The sample was kept at this temperature for three minutes. Then the sample was cooled at a rate of 10° C./min to −90° C. for PE (−60° C. for PP), and kept isothermally at that temperature for three minutes. The sample was next heated at a rate of 10° C./min, until complete melting (second heat). Unless otherwise stated, melting point (Tₘ) and the glass transition temperature (T_{g}) of each polymer were determined from the second heat curve, and the crystallization temperature (T_{c}) was determined from the first cooling curve. The respective peak temperatures for the Tₘ and the T_{c} were recorded. The percent crystallinity can be calculated by dividing the heat of fusion (H_{f}), determined from the second heat curve, by a theoretical heat of fusion of 292 J/g for PE (165 J/g for PP), and multiplying this quantity by 100 (for example, % cryst.=(Hf/292 J/g)×100 (for PE)). In DSC measurements, it is common that multiple Tₘ peaks are observed, and here, the highest temperature peak as the Tₘ of the polymer is recorded.

Table 3 provides information on the materials for making HFP1 and HFP2.

TABLE 3

| Component Name | Component Role | Component Source |
|---|---|---|
| Ethylene | Monomer | Airgas |
| 1-Octene | Comonomer | Delta Chemical Services |
| | | “AlphaPlus” Chevron |
| | | Phillips Chemical Company |
| 7-Octenyldimethylsilane | Silane | Gelest |
| | comonomer | |
| 5-Hexenyldimethylsilane | Silane | Gelest |
| | comonomer | |
| Toluene | Solvent | Sigma Aldrich |
| ISOPAR-E | Solvent | ExxonMobil |
| Zirconium, dimethyl[[2,2″′-[1,3- | Pre-catalyst | Disclosed in |
| propanediylbis(oxy- | | WO2007136497A2 |
| κO)]bis[3″,5,5″-tris(1,1- | | Synthesized according to |
| dimethylethyl)-5′-methyl[1,1′:3′,1″- | | procedure in |
| terphenyl]-2′-olato-κO]](2-)]- (ACI) | | US20040010103A1 |
| (CAS# 958647-88-6) | | |
| bis(hydrogenated tallow | Activator | Synthesis disclosed in |
| alkyl)methyl tetrakis(pentafluoro- | | U.S. Pat. No. 6,121,185A |
| phenyl)borate amine | | |
| MMAO-3A | Scavenger | AkzoNobel |

### Preparation of Crosslinked Elastomers and Elastomer Gel Pastes

Table 4 lists the components for use in preparing the crosslinked elastomers

TABLE 4

| Component | Description | Source |
|---|---|---|
| Diallyl propylene | CH₂═CHCH₂O[CH₂CH(CH₃)O]₂₀—CH₂CH═CH₂ | NOF Corporation under |
| glycol crosslinker | | the name Polycerin |
| | | DUS-80. |
| Catalyst | vinyl end-blocked polydimethylsiloxane | Available under the |
| | diluted platinum complex of 1,3-diethenyl- | name SYL-OFF ™ 4000 |
| | 1,1,2,2-tetramethyldisiloxane having a | from The Dow Chemical |
| | platinum concentration of 5,200 weight parts | Company |
| | per million | |

SYL-OFF is a trademark of Dow Silicones Corporation

Example 1. First, prepare a 10.0 wt % solution of HFP1 in isododecane by adding 10 g of HFP1 and 90 g of isododecane to a first glass jar, seal the jar and roll on a three mill roll for 48 hours to form a homogeneous solution. Then, into a second glass jar, add 29.71 g of the homogeneous solution from the first glass jar along with 0.56 g of diallyl propylene glycol crosslinker, and 29.71 g of isododecane. Add a stir cross. Place the jar into an 80° C. hot water bath and initiate stirring. Syringe 0.12 g of Catalyst into the jar once the contents reach a temperature of 75-80° C. and continue mixing until gelation occurs. Place the jar into a 70° C. over for 4 hours to achieve crosslinked elastomer Example 1.

Prepare an elastomer gel paste from Example 1 by placing 59.08 g of Example 1 into a Waring Blender and shearing for 20 seconds each on setting 1, 3 and 5 without scraping or stopping between settings. Add 5.91 g of isododecane and shear for 30 seconds each on setting 1,2, and 3. In between setting to ensure good mixing. The resulting material is an elastomer gel past of Example 1 having an average particle size of solvent swollen Example 1 crosslinked elastomer that is 45 micrometers.

Example 2. First, prepare a 10.0 wt % solution of HFP2 in isododecane by adding 10 g of HFP1 and 90 g of isododecane to a first glass jar, seal the jar and roll on a three mill roll for 48 hours to form a homogeneous solution. Then, into a second glass jar, add 20.21 g of the homogeneous solution from the first glass jar along with 0.38 g of diallyl propylene glycol crosslinker, and 9.41 g of isododecane. Add a stir cross. Place the jar into an 80° C. hot water bath and initiate stirring. Syringe 0.06 g of Catalyst into the jar once the contents reach a temperature of 75-80° C. and continue mixing until gelation occurs. Place the jar into a 70° C. oven for 4 hours to achieve crosslinked elastomer Example 2.

Prepare an elastomer gel paste from Example 2 by placing 26.25 g of Example 2 into a Waring Blender and shearing for 20 seconds each on setting 1, 3 and 5 without scraping or stopping between settings. Add 6.06 g of isododecane and shear for 30 seconds each on setting 1,2, and 3. In between setting to ensure good mixing. The resulting material is an elastomer gel paste of Example 2.

### Sensory Evaluation

Conduct a sensory evaluation of the elastomer gel paste of Example 2 and DOWSIL™ 9045 Silicone Elastomer Blend (DOWSIL is a trademark of The Dow Chemical Company). DOWSIL™ 9045 Silicone Elastomer Blend is recognized in the industry for having a desirably silky, smooth, and powdery sensory attributes and is used as a benchmark for sensory performance.

Conduct a sensory evaluation with four researchers familiar with crosslinked elastomer pastes and who have experience in performing sensory evaluation on skin. Have each researcher evaluate the elastomer gel paste of Example 2 and the DOWSIL™ 9045 Silicone Elastomer Blend in a blind test where one sample is identified as “Sample A” and the other as “Sample B”. Have the researchers assess seven attributes and rate each sample on a scale of 1 to 5 for each attribute. Table 5 presents the seven attributes, equivalent materials for a value of 1 and 5 for each attribute (values between 1 and 5 have a sensory character between that of the equivalent materials), an ideal value for the sensory character, and the average sensory value for each sample assigned by the four researchers:

TABLE 5

| | | | | DOWSIL ™ | |
|---|---|---|---|---|---|
| | 1 | 5 | | 9045 Silicone | |
| | (equivalent | (equivalent | Ideal | Elastomer | Example 2 |
| Attribute | material) | material) | Value | Blend | Paste |
| Wetness - perceived | Talc | Water | 3 | 3.25 | 3 |
| amount of water while | | | | | |
| rubbing | | | | | |
| Spreadability- ease of | Lanolin | Mineral Oil | 5 | 3 | 4 |
| moving the product over | | | | | |
| the skin | | | | | |
| Tackiness-degree to which | Untreated | Lanolin | 1 | 3 | 3.25 |
| finger sticks to skin | skin | | | | |
| Greasiness-tacky, dense | Talc | Petrolatum | 1 | 3 | 3.25 |
| coating, frequently with | | | | | |
| drag | | | | | |
| Slipperiness-ease of | Lanolin | Mineral oil | 5 | 3 | 2.5 |
| moving fingers across skin | | | | | |
| Smoothness-an even | Sand | Talc | 5 | 3 | 2.75 |
| surface film; uniformity of | | | | | |
| texture | | | | | |
| Powderiness- dry soft, | Petrolatum | Talc | 5 | 3 | 2 |
| smooth, slippery feel | | | | | |

The paste of Example 2 performs similar and in some cases better than the DOWSIL™ 9045 Silicone Elastomer Blend in the sensory attributes.

### Organic Solvent Compatibility

Evaluate solvent compatibility of the elastomer gel paste of Example 1 (Example 1 paste) against DOWSIL™ 9045 Silicone Elastomer Blend and DOWSIL™ EL-8040 ID Silicone Organic Blend with 7 different organic solvents by mixing 8-10 gram batches of 25 wt % of Example 1 paste or, DOWSIL™ 9045 Silicone Elastomer Blend or DOWSIL™ EL-8040 ID Silicone Organic Blend in the solvent, mix on a speed mixer for one minute and then transfer to a clear vial. Allow the samples to set for 12 hours at 25° C. and then evaluate the samples for clarity and relative thickening according to the evaluation ranking below. Table 6 lists the seven solvents and results for Example 1 and DOWSIL™ 9045 Silicone Elastomer Blend.

### Clarity Ranking

A Clear
B Slightly Hazy
C Hazy
D Cloudy
E Opaque
F Not Compatible—inhomogeneous mixture

### Viscosity Ranking

Evaluate viscosity by inverting the vial and observing how the mixture flows:
1 Liquid (comparable to DOWSIL™ 245 fluid)
3 Pourable (comparable to 10,000 square millimeters per second (centiStoke) polydimethylsiloxane)
5 Pourable (comparable to 60,000 square millimeters per second (centiStoke) polydimethylsiloxane)
9 Barely Pourable (comparable to 300,000 square millimeters per second (centiStoke) polydimethylsiloxane)
10 Does not flow when inverted.

TABLE 6

| | DOWSIL ™ 9045 | | DOWSIL ™ 8040 | | | |
|---|---|---|---|---|---|---|
| | Silicone Elastomer | | Silicone Organic | | | |
| | Blend | | Elastomer Blend | | Example 1 paste | |
| | Gel Paste Solvent: | | Gel Paste Solvent: | | Gel Paste Solvent: | |
| | cylcopentasiloxane | | isododecane | | isododecane | |
| Organic Solvent | Clarity | Viscosity | Clarity | Viscosity | Clarity | Viscosity |
| Octylmethoxycinnamate | E | 4 | D | 3 | A | 8 |
| Ethylhexyl Salicylate | C | 9 | C | 10 | B | 8 |
| Crodamol ™ AB emollient | F | NM* | C | 10 | B | 8 |
| Crodamol ™ GTCC mixed | F | NM* | C | 4 | C | 4 |
| ester | | | | | | |
| Sunflower Oil | D | 2 | F | NM* | B | 5 |
| Squalane | C | 2 | C | 2 | C | 5 |
| Mineral Oil | D | 3 | C | 10 | C | 7 |

Crodamol is a trademark of Croda, INC.
*NM = not measured due to inhomogeneous mixture.

The inventive material has a broader compatibility with the solvents than either DOWSIL™ 9045 Silicone Elastomer Blend or DOWSIL™ 8040 Silicone Organic Elastomer Blend as is evident by achieving a homogenous mixture with all seven solvents. The inventive material also thickens across more of the solvents than the DOWSIL™ 9045 Silicone Elastomer Blend.

### Oil in Water Formulation

Evaluate the ability to form an oil-in-water system using Example 1 paste versus DOWSIL™ 9045 Silicone Elastomer Blend.

Form an initial mixture by combining together the following components and then mix with a marine propeller at 500 revolutions per minute (RPM), stopping to scrape sides/bottom of the beaker with a spatula as needed, until achieving a uniform mixture: 2 g XIAMETER™ PMX-200 silicone Fluid (200 square millimeters per second (centiStokes) viscosity), 5 g XIAMETER PMC-0245 cyclopentasiloxane, 3.8 g of Dow Corning RM 2051 thickening agent, and 10 g of either Example 1 paste or and DOWSIL™ 9045 Silicone Elastomer Blend. XIAMETER is a trademark of Dow Corning Corporation.

Combine in a separate vessel 74 g water and 2 g of glycerin. Add the resulting mixture to the initial mixture as a constant stream while mixing, increasing mixing rate to 1000 RPM. Add 3 g DOWSIL™ 9509 silicone elastomer suspension (DOWSIL is a trademark of The Dow Chemical Company) while mixing and then 0.2 g of GLYDANT™ Plus preservative (GLYDANT is a trademark of Lonza, LLC).

Both formulations with Example 1 paste and DOWSIL™ 9045 Silicone Elastomer Blend result in white lotions demonstrating capability of easily forming an oil-in-water system. Both formulations have similar viscosities and feel on the skin when rubbed in.

### Clear Sunscreen Formulation

Prepare clear sunscreen formulations using elastomer gel paste of Example 2 (Example 2 paste) and another using DOWSIL™ 9045 Silicone Elastomer Blend to compare results.

Into a speedmixer cup add 67.45 g of either Example 2 paste or and DOWSIL™ 9045 Silicone Elastomer Blend. While mixing at 2300 RPM add each of the following components in order while mixing for one minute after each addition to achieve a homogeneous mixture: 7.5 g octyl methoxycinnamate, 5 g ethylhexyl salicylate, 8 g Crodamol™ GTCC mixed ester, 12 g Cetiol™ OE emollient (Cetiol is a trademark of Cognis IP management GMBH), and 0.05 g of DOWSIL™ VM-2270 aerogel fine particles (average particle size of 5-15 micrometers, surface area of 600-800 square meters per gram and porosity of greater than 90).

Both formulations are colorless. However, only the one with Example 2 paste is clear and stable. The formulation using DOWSIL™ 9045 Silicone Elastomer Blend is cloudy and not stable. This demonstrates the ability of the inventive material to prepare clear sunscreen formulations even better than DOWSIL™ 9045 Silicone Elastomer Blend.

What is claimed is:

1. A composition comprising a crosslinked elastomer comprising polyolefin backbones crosslinked through polyether chains wherein the crosslinked elastomer has a carbon-silicon-carbon linkage between the polyolefin backbone and polyether chain and wherein the crosslinked elastomer is free of Si—O—Si bonds.

2. The composition of claim 1, wherein the polyolefin backbone is an interpolymer of ethylene, octene, and at least one of 7-octenyldimethylsilane and 5-hexenyldimethylsilane and wherein the polyether chains crosslink by bonding to the silane.

3. The composition of claim 2, wherein the polyolefin backbone is an interpolymer that consists of the following polymerized monomer segments:
(a) 20 to 35 mol-percent of octene;
(b) 65 to 80 mole-percent ethylene; and
(c) 1 to 10 mole-percent of one or any combination of more than one of 7-octenyldimethylsilane and 5-hexenyldimethylsilane.

4. The composition of claim 1, wherein the polyether chains comprise polyether units selected from —R—O— groups where R is an alkylene having from 2 to 8 carbon atoms.

5. The composition of claim 4, where the polyether chain comprises polypropylene oxide.

6. The composition of claim 1, wherein the crosslinked elastomer consists of the following interpolymerized polymer segments:
(a) 20 to 35 mole-percent of one more a combination of more than one of segment with the average chemical structure —CH₂CHR′—;
(b) 65 to 80 mole-percent —CH₂CH₂—; and
(c) 1 to 10 mole-percent of —CH₂CHA-;
wherein mole-percent is relative to moles of interpolymerized polymer segments in the crosslinked elastomer, R′ is an alkyl group having one or more and at the same time 8 or fewer carbon atoms and A has the following average chemical structure (I):
—(CH₂)ₙSi(CH₃)₂—B-D (I)
and where
n is a value in a range of 4 to 6;
B is —(CH₂)₃O(RO)_{p′}(CH₂)₃—; where R is an alkylene having 2 or more and at the same time 8 or fewer carbon atoms and subscript p′ has a value in a range of 15-25; and
D is a —Si(CH₃)₂(CH₂)ₙ— group of another A group.

7. The composition of claim 1, wherein the composition further comprises a solvent and the crosslinked elastomer is solvent swollen so as to be a solvent swollen elastomer.

8. The composition of claim 7, wherein the solvent swollen elastomer has an average particle size in a range of one to 500 micrometers.

9. A process for producing the composition of claim 1, the process comprising providing a crosslinked elastomer by conducting a hydrosilylation reaction with a reaction composition comprising:
(a) a hydrosilane functional polyolefin that is free of Si—O—Si bonds;
(b) a polyether crosslinker comprising at least two terminal alkenyl groups and that is free of Si—O—Si bonds;
(c) a hydrosilylation catalyst; and
(d) an organic solvent.

10. The process of claim 9, wherein the hydrosilane functional polyolefin is an interpolymer of:
(a) 20 to 35 mole-percent 1-octene;
(b) 65 to 80 mole-percent of ethylene; and
(c) one to 10 mole-percent of one or any combination of both of 7-octenyldimethylsilane and 5-hexenyldimethylsilane with mole-percent relative to total moles of 1-octene, ethylene, 7-octenyldimethylsilane and 5-hexeneyldimethylsilane.
